(19) United States
(12) Patent Application Publication
(10) Pub. No.: US 2005/0080415 A1
(43) Pub. Date: Apr. 14, 2005

(54) Polyaxial bone anchor and method of spinal fixation

(76) Inventors: Thomas R. Keyer, Aston, PA (US); Roger Berger, Buren (CH); Martin Walther, West Chester, PA (US)

Correspondence Address: JONES DAY, 51 Louisiana Aveue, N.W, WASHINGTON, DC, 20001-2113, US

(21) Appl. No.: 10/682,999

(22) Filed: Oct. 14, 2003

(51) Int. Cl. A61B 17/70; A61B 17/86

(52) U.S. Cl. 606/61; 606/73

(57) ABSTRACT

The present invention is directed to a polyaxial bone anchor for attaching a rod to a bone comprising an anchor member for attachment to the bone, a body member having a U-shaped channel for receiving the rod and a compressible recess for receiving a head of the anchor member such that the anchor member can initially polyaxially angulate with respect to the body member, a collar slidably disposed about the body member and capable of compressing the recess around the head, and a fastener capable of pressing the rod against the collar. The body member may define a first axis, an upper bounding edge, and a lower bounding edge, and the lower bounding edge may include a countersunk region to permit increased angulation of the anchor member with respect to the first axis when the anchor member is oriented toward the countersunk region. Other structures for providing increased angulation of the anchor member are disclosed as well. Further, the present invention is directed to methods of fixation of the cervical region of the spine.

### TECHNICAL FIELD OF THE INVENTION

The present invention relates generally to bone fixation devices and related methods of fixation. More specifically, the present invention relates to polyaxial bone anchors, such as screws and hooks for spinal fixation, and related methods of spinal fixation.

### BACKGROUND OF THE INVENTION

There are many methods of treating spinal disorders known in the art. One known method involves anchoring a screw or a hook to the vertebrae, and fixing the screws or hooks along a spinal rod to position or immobilize the vertebrae with respect to one another. The screws or hooks commonly have heads with U-shaped channels that the spinal rod is inserted into and subsequently clamped into by a set screw or other fastener mechanism. This method may commonly involve multiple screws or hooks, as well as multiple spinal rods. With this method, the spinal rod(s) may be shaped to maintain the vertebrae in such an orientation as to correct the spinal disorder at hand (e.g., to straighten a spine having abnormal curvature). Additionally or alternatively, the screws or hooks may be spaced along the rods(s) to compress or distract adjacent vertebrae.

Surgeons have often encountered considerable difficulty when using this method, due to trouble aligning the spinal rod(s) with the U-shaped channels in the heads of the screws or hooks. For example, the heads of the screws or hooks are often out of alignment with one other due to the curvature of the spine or the size and shape of each vertebrae. In order to facilitate easier insertion of the spinal rods into the U-shaped channels, and to provide additional flexibility in the positioning of the spinal rods and the screws and hooks, screws and hooks have been developed with which the head or “body” (and consequently the U-shaped channel) initially pivots with respect to the screw shank or the hook. One example of such a screw is disclosed in U.S. Pat. No. 5,586,984 to Errico et al., the content of which is incorporated herein by reference. The device disclosed in the Errico patent, and other similar known devices, typically allow symmetrical angulation of the screw or hook with respect to the body. One limitation with these devices, however, is that the degree of angulation can be limited due to contact between the shank of the screw or hook, and the lower bounding edge of the body. This can be problematic in certain spinal applications where increased angulation is required, for example, in treatment of the cervical region of the spine.

Therefore, there remains a need in the art for polyaxial bone anchors that provide increased angulation between the head and the screw or hook. There also remains a need in the art for methods of treating spinal disorders that require increased angulation, such as fixation of the cervical region of the spine.

### SUMMARY OF THE INVENTION

The present invention is directed to a polyaxial bone anchor for attaching a rod, such as a spinal rod, to a bone, such as a vertebra. The polyaxial bone anchor may include an anchor member (such as a screw or a hook) for attachment to the bone, a body member having a U-shaped channel for receiving the rod and a compressible recess for receiving a head of the anchor member such that the anchor member can initially polyaxially angulate with respect to the body member, a collar slidably disposed about the body member and capable of compressing the recess around the head, and a fastener capable of pressing the rod against the collar. The body member may define a first axis, an upper bounding edge, and a lower bounding edge, and the lower bounding edge may include a countersunk region to permit increased angulation of the anchor member with respect to the first axis when the anchor member is oriented toward the countersunk region. The bounding edge may be configured and dimensioned to permit the anchor member to angulate through a first angle of about 30° with respect to the first axis, and the countersunk region may be configured and dimensioned to permit the anchor member to angulate through a second angle of about 50° with respect to the first axis. Alternatively, the first angle may be about 20° and the second angle may be about 45°. The countersunk region may extend through an angular region of between about 5° and about 180° with respect to the first axis. Preferably, the countersunk region may extend through and angular region of between about 15° and about 20° with respect to the first axis. The U-shaped channel may define a second axis, and a midpoint of the countersunk region may be offset from the second axis by about +/−45° or less. According to one exemplary embodiment, the midpoint of the countersunk region may be offset from the second axis by between about 20° and about 25° (in the positive or negative direction). At least a portion of the body member may have a tapered exterior surface, and at least a portion of the collar may have a tapered interior surface. Sliding the collar downward with respect to the body member, for example by tightening the fastener against the rod, may cause the tapered interior surface to engage the tapered exterior surface to compress the recess around the head to fix the orientation of the anchor member with respect to the body member.

According to another embodiment of the present invention, the polyaxial bone anchor may include an anchor member for attachment to the bone, a body member polyaxially mounted to the anchor member, a seat for orienting the rod, and a fastener capable of engaging the body member to press the rod against the seat. The body member may define a first axis, and the seat may orient the rod along a second axis, wherein the first axis is oriented at an acute angle with respect to the second axis. For example, the first axis may be oriented at an angle of between about 60° and about 40° with respect to the second axis. Alternatively, the first axis may be oriented at an angle of between about 70° and about 45° with respect to the second axis. The polyaxial bone anchor may further include an insert member disposed within the body member for receiving the head, and the seat may be associated with the insert member. For example, the seat may define an inclined surface on the insert member that extends substantially parallel to the second axis. Alternatively or additionally, the bone anchor may further include a collar disposed around the body member, and the seat may be associated with the collar. For example, the seat may define an inclined surface on the collar that extends substantially parallel to the second axis.

According to another embodiment of the invention, the anchor member may include a bone screw having a shank with a first end attached to the head and a second end opposite the first end, and the shank may include an unthreaded portion and a threaded portion. The unthreaded portion is preferably substantially adjacent to the first end, and the threaded portion is preferably substantially adjacent to the second end. The shank may define a shank length from the first end to the second end, and the unthreaded portion may extend over greater than about ¼ of the shank length. Preferably, the unthreaded portion extends over greater than about ½ of the shank length. Additionally or alternatively, the unthreaded portion may define an unthreaded outer diameter, and the threaded portion may define an inner thread diameter and an outer thread diameter, wherein the outer thread diameter is greater than the unthreaded outer diameter. Also, the unthreaded outer diameter may be greater than the inner thread diameter. Alternatively, the unthreaded outer diameter may be equal to or less than the inner thread diameter.

The present invention is also related to a method of fixating the cervical region of the spine using a first polyaxial bone anchor having a first screw member and a first body member with a first rod-receiving channel, and a second polyaxial bone anchor having a second screw member and a second body member with a second rod-receiving channel. The method may include the steps of inserting the first screw member through a first vertebra and into a second vertebra, inserting the second screw member into a third vertebra, aligning the first rod-receiving channel with the second rod-receiving channel, and securing a spinal rod in the first rod-receiving channel and in the second rod-receiving channel. The first screw member may extend through the C2 vertebra and into the C1 vertebra. For example, the first screw member may extend through a claudal articular process of the C2 vertebra and into a lateral mass of the C1 vertebra, thereby immobilizing the C1 vertebra with respect to the C2 vertebra. The first screw member may be inserted at an orientation of between about 0° and about 25° medially or laterally, and preferably between about 0° and about 15° medially or laterally. The first screw member may also be inserted at an orientation of between about 30° and about 50° upward, and preferably between about 30° and about 40° upward. The second screw member, for example, may be inserted into anyone of the vertebrae C3-C7, T1-T3.

According to another embodiment of the method, the first screw member may be inserted into a lateral mass of a first vertebra. A second screw member may be inserted into a lateral mass of a second vertebra. At least one of the first and second vertebrae may be selected from the group of vertebrae consisting of C3-C7 and T1-T3. The first screw member may be inserted at an orientation of between about 0° and about 45° laterally and between about 0° and about 50° upward. Preferably, the first screw member may be inserted at an orientation of between about 25° and about 45° upward.

### BRIEF DESCRIPTION OF THE DRAWINGS

The detailed description will be better understood in conjunction with the accompanying drawings, wherein like reference characters represent like elements, as follows:

FIG. 1 is a perspective view of a first illustrative embodiment of a polyaxial bone anchor according to the present invention;

FIG. 2 is a side view of the polyaxial bone anchor of FIG. 1;

FIG. 3 is a cross-sectional view of the polyaxial bone anchor of FIG. 1, taken along lines III-III of FIG. 2;

FIG. 4 is a side view of a body member of the polyaxial bone anchor of FIG. 1;

FIG. 5 is a top view of the body member of FIG. 4;

FIG. 6 is a side view of the polyaxial bone anchor of FIG. 1, shown with the anchor member angulated through a first angle;

FIG. 7 is a side view of the polyaxial bone anchor of FIG. 1, shown with the anchor member angulated through a second angle;

FIG. 8 is a side view of a second illustrative embodiment of a polyaxial bone anchor according to the present invention;

FIG. 9 is a side view of the polyaxial bone anchor of FIG. 8, with hidden portions shown in broken lines;

FIG. 10 is a side view of a third illustrative embodiment of a polyaxial bone anchor according to the present invention;

FIG. 11 is a side view of the polyaxial bone anchor of FIG. 10, with hidden portions shown in broken lines;

FIG. 12 is a side view of one illustrative embodiment of a set screw for securing a rod to a polyaxial bone anchor according to the present invention, with hidden portions shown in broken lines;

FIG. 13 is a top view of the set screw of FIG. 12;

FIG. 14 is a side view of one illustrative embodiment of a nut for securing a rod to a polyaxial bone anchor according to the present invention;

FIG. 15 is a bottom view of the nut of FIG. 14;

FIG. 16 is a side view of a fourth illustrative embodiment of a polyaxial bone anchor according to the present invention;

FIG. 17 is a side view of a fifth illustrative embodiment of a polyaxial bone anchor according to the present invention;

FIG. 18 is a cross-sectional view of the polyaxial bone anchor of FIG. 17, taken along line XVIII-XVIII;

FIG. 19 is a left lateral view of the cervical and upper thoracic regions of the spine, shown being stabilized by a first illustrative method of spinal fixation according to the present invention;

FIG. 20 is a posterior view of FIG. 19;

FIG. 21 is a left lateral view of the cervical and upper thoracic regions of the spine, shown being stabilized by a second illustrative method of spinal fixation according to the present invention; and

FIG. 22 is a posterior view of FIG. 21.

### DETAILED DESCRIPTION OF THE PREFERRED EMBODIMENTS

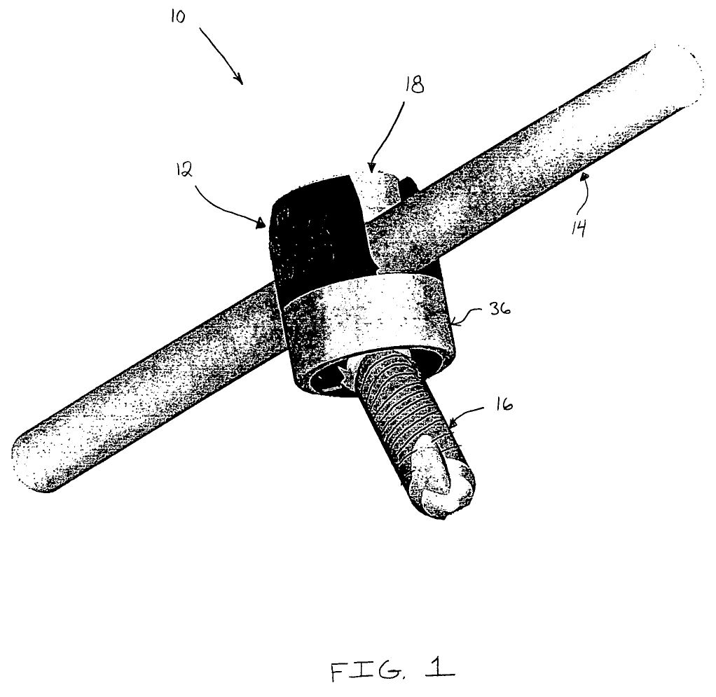

Referring to FIG. 1, a first illustrative embodiment of a polyaxial bone anchor according to the present invention is shown. Polyaxial bone anchor 10 generally includes a body 12 having a channel for receiving a spinal rod 14 or other device, an anchor member 16 attached to body 12 such that it can polyaxially rotate with respect to body 12, and a fastener 18 for securing the spinal rod 14 to body 12. Fastener 18 may also fix the angular position of anchor member 16 with respect to body 12. One or more polyaxial bone anchors 10 may be attached to the vertebrae via anchor member 16 (shown as a bone screw) and positioned along the spinal rod 14, or other device, to correctly align the spine or treat other spinal disorders.

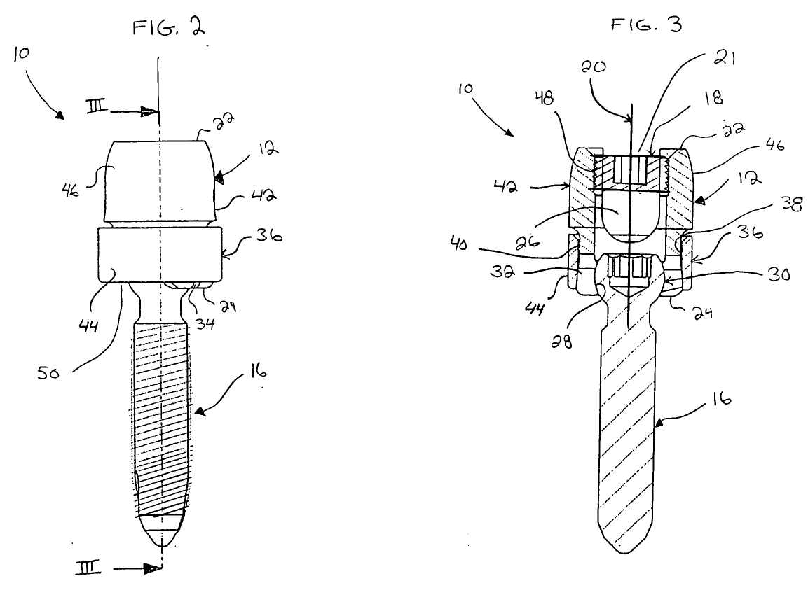

Referring to FIGS. 2 and 3, side and cross-sectional views of polyaxial bone anchor 10 are shown, respectively. As shown, body 12 may comprise a generally cylindrical member defining a first axis 20, an upper bounding edge 22 and a lower bounding edge 24. Body 12 may be substantially hollow or, in other words, define a bore 21 from the upper bounding edge 22 to the lower bounding edge 24. First axis 20 may extend along the center line of bore 21. Body 12 may include a rod-receiving channel 26 (shown for illustrative purposes as a U-shaped channel) formed in communication with the upper bounding edge 22 and/or the bore 21. A recess 28 may be formed substantially adjacent the lower bounding edge 24. In the illustrative embodiment shown, rod-receiving channel 26 is oriented substantially transversely to first axis 20, however other configurations are possible, as discussed below. Referring specifically to FIG. 3, anchor member 16 may include a curvate head 30 that is shaped and dimensioned to fit within recess 28 such that body 12 may polyaxially angulate on anchor member 16. As shown in the illustrative embodiment of FIG. 3, curvate head 30 may be substantially spherical or frustospherical, and recess 28 may be of a matching shape, however other shapes and configurations are contemplated. Curvate head 30 preferably has a recess that is keyed to receive a hex wrench, torx wrench, or other driver known in the art, to allow anchor member 16 to be implanted into a vertebra.

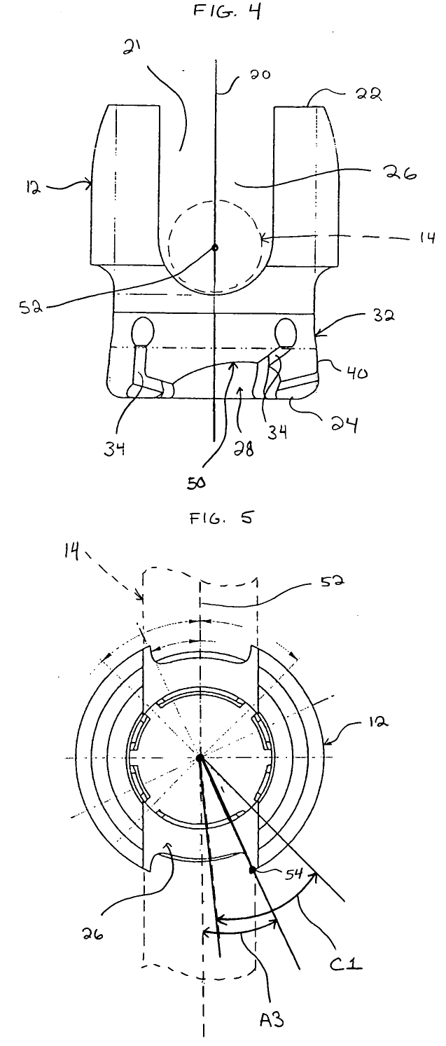

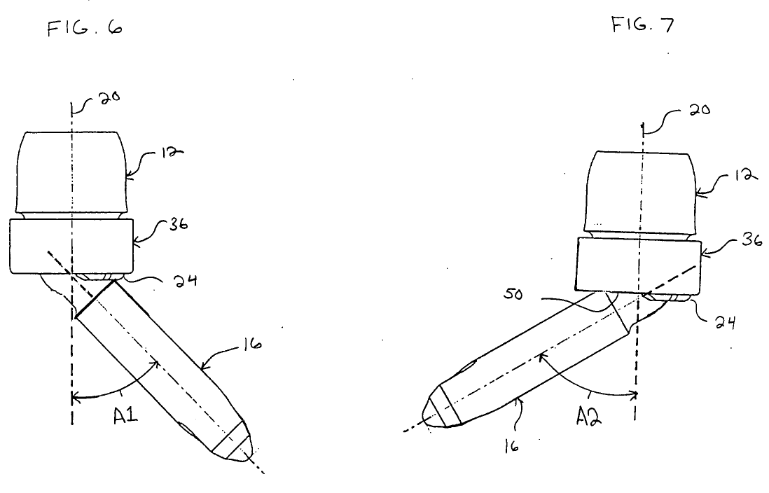

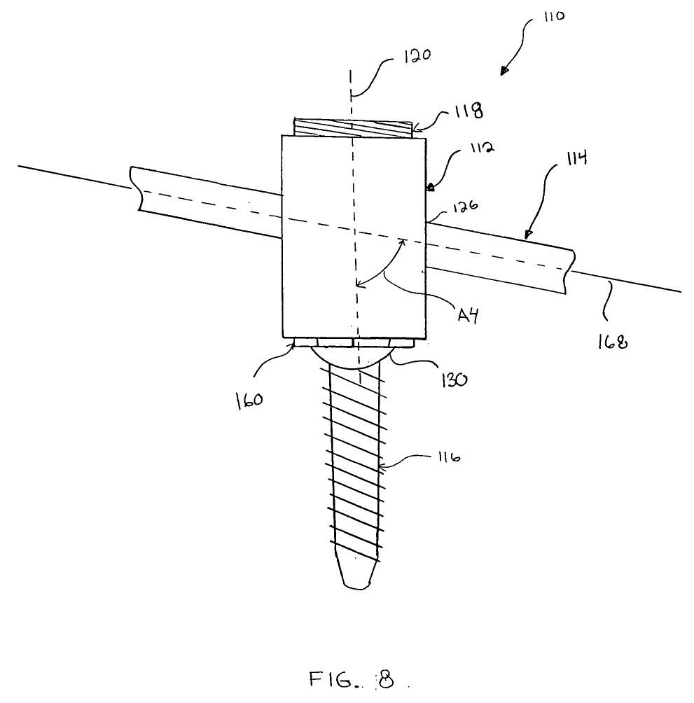

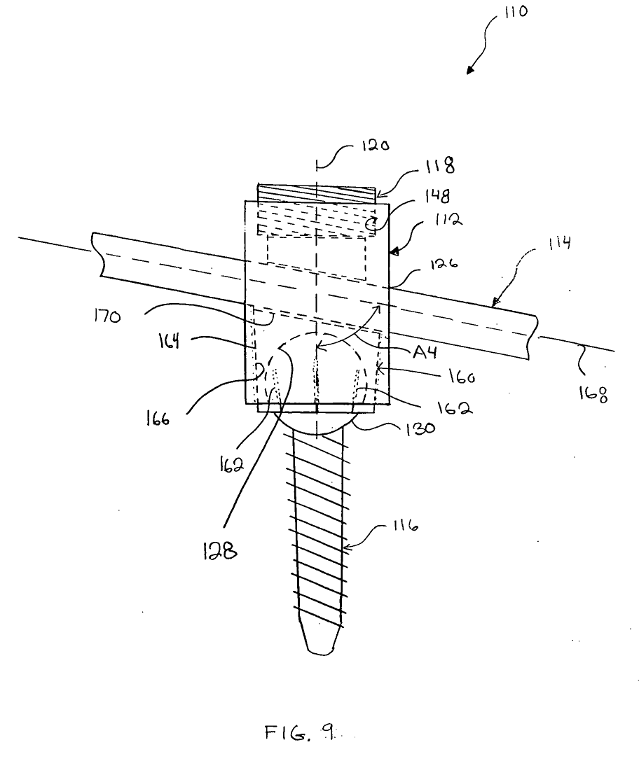

Referring to FIG. 4 in combination with FIGS. 2 and 3, the lower portion 32 of body 12 surrounding recess 28 is preferably compressible or resilient to allow body 12 to be snapped over curvate head 30. In the illustrative embodiment shown, lower portion 32 of body 12 has a plurality of slits 34 formed therein to provide the desired compressibility or resilience.

Still referring to FIGS. 2, 3 and 4, a collar 36 may be slidably disposed around the lower portion 32 of body 12. Collar 36 may have an inner surface 38 that interacts with the exterior surface of the lower portion 32 of body 12 to compress recess 28 around curvate head 30 when collar 36 is pressed downward with respect to body 12. More specifically, the inner surface 38 of collar 36 may be tapered, and/or the exterior surface 40 of the lower portion 32 of body 12 may be tapered. The exterior surface 40 of the lower portion 32 of body 12 may also be recessed inward with respect to the exterior surface of the upper portion 42 of body 12, such that the exterior surface 44 of collar 36 and the exterior surface 46 of the upper portion 42 of body 12 are of relatively the same diameter. This configuration may help minimize the profile of polyaxial bone anchor 10.

Fastener 18, shown in FIG. 3 as a set screw, may engage internal threads 48 formed on the inside surface of the upper portion 42 of body member 12. Tightening fastener 18 onto body 12 moves the fastener 18 against spinal rod 14 (when located in the rod-receiving channel 26) and urges spinal rod 14 against collar 36, in turn causing collar 36 to slide downward along the tapered exterior surface 40 of lower portion 32 of body 12. Consequently, lower portion 32 contracts recess 28 around the curvate head 30 of anchor member 16, and locks the angular position of anchor member 16 with respect to body 12. In other words, tightening fastener 18 sufficiently prevents polyaxial movement of anchor member 16 with respect to body 12. In addition, the opposing forces applied on spinal rod 14 by fastener 18 and collar 36 fixes the position and orientation of spinal rod 14 on body 12. The collar 36 and body 12 may be configured such that loosening the fastener 18 after the anchor member 16 and spinal rod 14 were previously fixed in position may allow a user to move and reposition spinal rod 14 in channel 26 while the anchor member 16 remains fixed with respect to the body 12. For example, the collar 36 and body 12 may be provided with substantially matching or corresponding tapers. According to this configuration, the anchor member 16 may require the user to actively unlock it, by for instance, the use of a release instrument, in order for the anchor member 16 to once again polyaxially angulate with respect to body 12. While fastener 18 is shown in FIG. 3 as an internal set screw, other embodiments are contemplated by the present invention, including those discussed below.

Referring to FIGS. 4 and 5, body 12 may be adapted and configured to permit increased angulation of anchor member 16 with respect to body 12 over certain angular regions. Body 12, and more specifically bounding edge 24, may include a recessed or countersunk region 50. Due to the configuration of countersunk region 50, anchor member 16 can angulate through a greater angle with respect to first axis 20 before contacting lower bounding edge 24 when it is oriented towards countersunk region 50, than it can when anchor member is oriented away from countersunk region 50 (i.e., towards the remaining portions of lower bounding edge 24). As shown in FIG. 6, lower bounding edge 24 may be dimensioned and configured to provide angulation of anchor member 16 though a first angle A1 before anchor member 16 contacts lower bounding edge 24. As shown in FIG. 7, countersunk region 50 (hidden in part by collar 36) may be dimensioned and configured to provide angulation of anchor member 16 through a second angle A2 before further angulation is stopped by contact between anchor member 16 and countersunk region 50 or collar 36. According to one preferred embodiment, first angle A1 may be about 30° (permitting anchor member 16 to angulate between about 0° and about 30°) and second angle A2 may be about 50° (permitting anchor member 16 to angulate between about 0° and about 50°). According to another preferred embodiment, first angle A1 may be about 20° and second angle A2 may be about 45°.

Referring back to FIGS. 4 and 5, countersunk region 50 may be oriented with respect to rod-receiving channel 26, and consequently spinal rod 14 (shown in broken lines) to suit different medical applications. As shown, spinal rod 14 (when located in the rod-receiving channel 26) defines a second axis 52. Countersunk region 50 defines a midpoint 54. Midpoint 54 may be angularly offset from second axis 52 by a third angle A3 of about +/−45° or less. More preferably, third angle is between about 20° and about 25° (in the positive or negative direction). According to the illustrative embodiment shown in FIGS. 4 and 5, third angle A3 is approximately 22.5°, although other angles and configurations are possible. Countersunk region 50 may extend through an angular region C1 of between about 5° and about 180°, and preferably between about 15° and about 20°, although other angles and configurations are possible.

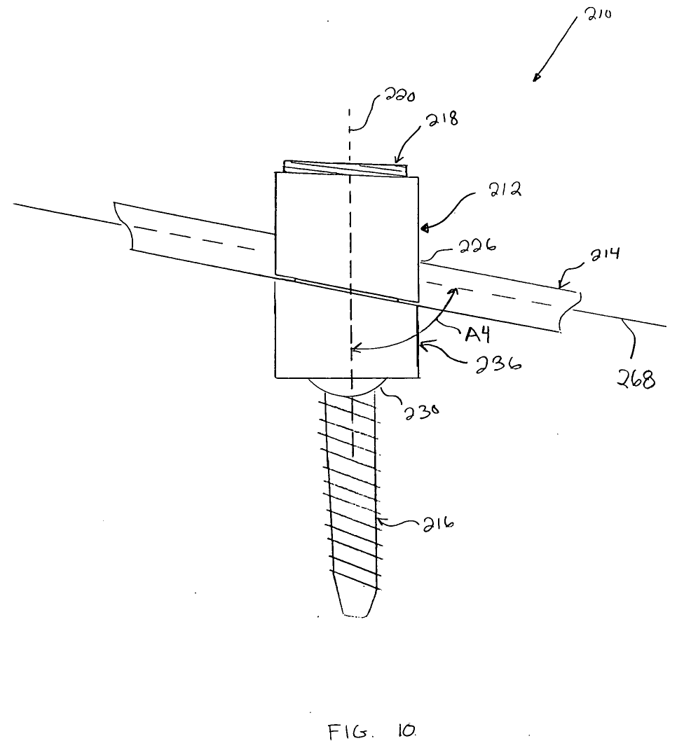

Referring to FIGS. 8 and 9, a second illustrative embodiment of a polyaxial bone anchor is shown. Polyaxial bone anchor 110 generally includes a body 112 having a rod-receiving channel 126 for receiving a spinal rod 114, an anchor member 116 (shown for illustrative purposes as a bone screw) having a curvate head 130, and a fastener 118. Body 112 may define a first axis 120. Polyaxial bone anchor 110 may also include an insert member 160 that is slidably disposed within body 112 and has a recess 128 for receiving the curvate head 130 of anchor member 116. Recess 128 and/or curvate head 130 are preferably configured and dimensioned such that anchor member 116 may polyaxially angulate with respect to insert member 160 and consequently body 112. For example, curvate head 130 and recess 128 may be spherical or frustospherical, as shown in FIGS. 8 and 9.

Still referring to FIGS. 8 and 9, insert member 160 is preferably compressible around curvate head 130. For example, a plurality of slits 162 may be provided in insert member 160, although other known configurations for providing the desired compressibility may alternatively be implemented. For example, insert member 160 may be formed of a resilient material. In addition, insert member 160 may have an exterior tapered surface 164, and/or body 112 may have a corresponding interior tapered surface 166. The corresponding tapered surfaces 164, 166 may serve to compress insert member 160 and recess 128 about curvate head 130 when insert member 160 is pressed downward within body 112 (e.g., by the force of spinal rod 114); thereby fixing the angular position of anchor member 116 with respect to insert member 160 and body 112. As shown in FIGS. 8 and 9, fastener 118 may be an internal set screw that engages internal threads 148 formed on body 112, although other configurations of fastener 118 are possible, including those discussed below.

Tightening fastener 118 presses spinal rod 114 against insert member 160 and causes insert member 160 to move downward in body 112. Consequently, tightening fastener 118 fixes the angular position of anchor member 116 with respect to body 112, and also secures spinal rod 114 in rod-receiving channel 126. The insert member 160 and body 112 may be configured such that loosening the fastener 118 after the anchor member 116 and spinal rod 114 have been fixed in position allows a user to move spinal rod 114 in channel 126 while the anchor member 116 remains fixed with respect to the body 112. For example, the insert member 118 and body 112 may be provided with substantially matching or corresponding tapers. According to this configuration, the anchor member 116 may require the user to actively unlock it by, for instance, the use of a release instrument in order for the anchor member 116 to once again polyaxially angulate with respect to body 112.

Polyaxial bone anchor 110 may be configured such that the spinal rod 114 extends along a second axis 168 that is oriented at an acute angle A4 with respect to the first axis 120 of body 112. For example, a seat 170 may be provided on insert member 160 to orient spinal rod 114 along the second axis 168. Seat 170 may be an inclined surface formed on the upper portion of insert member 160. Preferably, seat 170 extends substantially parallel to second axis 168. Alternatively, seat 170 may be provided on body 112 itself, for example, by angling the rod-receiving channel 126 with respect to first axis 120. In other words, the two U-shaped cutouts in body 112 that form the rod-receiving channel 126 will be of different sizes. According to one preferred embodiment, angle A4 is between about 40° and about 60°. According to another preferred embodiment, angle A4 is between about 45° and about 70°, although other angles are possible. Additionally, body 112 and/or insert member 160 may be provided with a countersunk region, as described above with respect to FIGS. 1-9.

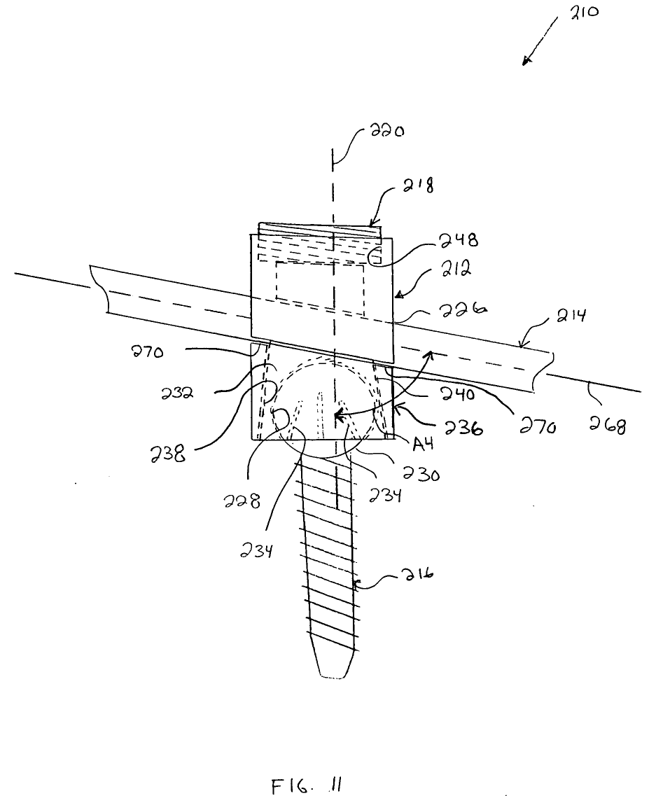

Referring to FIGS. 10 and 11, a third illustrative embodiment of a polyaxial bone anchor is shown. Polyaxial bone anchor 210 generally includes a body 212 having a rod-receiving channel 226 for receiving spinal rod 214, an anchor member 216 (shown for illustrative purposes as a bone screw) having a curvate head 230, and a fastener 218 for securing spinal rod 214 in the rod-receiving channel 226. Body 212 may define a first axis 220. Polyaxial bone anchor 210 may also include a collar 236 slidably disposed around the lower portion 232 of body 212.

As was the case with polyaxial bone anchor 10 (shown in FIGS. 1-7), body 212 may have a recess 228 for receiving curvate head 230 such that anchor member 216 can polyaxially angulate with respect to body 212. Preferably, recess 228 and curvate head 230 are substantially spherical or frustospherical, although other configurations are possible. Also, the lower portion 232 of body 212 preferably has a plurality of slits 234 that allow body 212 and recess 228 to compress about curvate head 230. Slits 234 may also allow body 212 to resiliently snap onto curvate head 230. In addition, the inner surface 238 of collar 236 and/or the outer surface 240 of lower portion 232 of body 212 may have matching tapers that cause body 212 and recess 228 to compress around curvate head 230 when collar 236 is moved downward with respect to body 212. Thus, tightening fastener 218 against spinal rod 214 moves collar 236 downward against collar 236 to compress body 212 and recess 228 about curvate head. Consequently, the angular position of anchor member 216 is fixed with respect to body 212, and spinal rod 214 is secured in rod-receiving channel 226. The collar 236 and body 212 may be configured such that loosening the fastener 218 after the anchor member 216 and spinal rod 214 have been fixed in position allows a user to move spinal rod 214 in channel 226 while the anchor member 216 remains fixed with respect to the body 212. For example, the collar 236 and body 212 may be provided with substantially matching or corresponding tapers. According to this configuration, the anchor member 216 may require the user to actively unlock it by, for instance, the use of a release instrument in order for the anchor member 216 to once again polyaxially angulate with respect to body 212.

Collar 236 may include a seat 270 that orients spinal rod 214 along a second axis 268. Seat 270 may comprise the inclined upper surface of collar 236 that contacts spinal rod 214 when located in the rod-receiving channel 226, in which case, the inclined upper surface is preferably parallel to second axis 268. According to one preferred embodiment, seat 270 positions spinal rod 214 such that the second axis 268 forms an acute angle A4 with the first axis 220 of body 212. According to one preferred embodiment, angle A4 may be between about 40° and about 60°. According to another preferred embodiment, angle A4 may be between about 45° and about 70°, although other angles are possible. Body 212 and/or collar 236 may also be provided with a countersunk region, such as described above with respect to FIGS. 1-9.

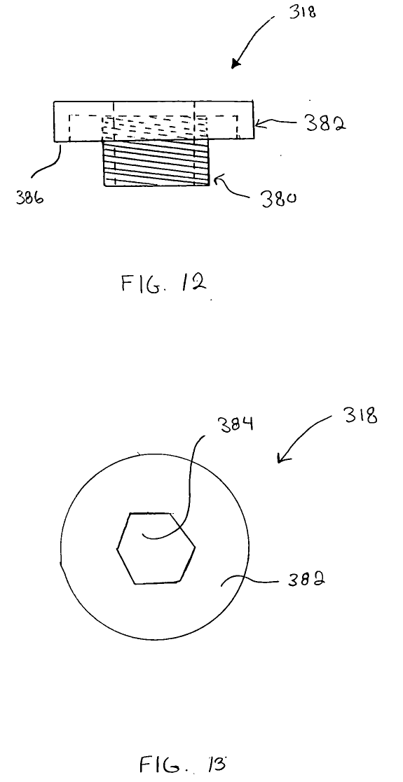

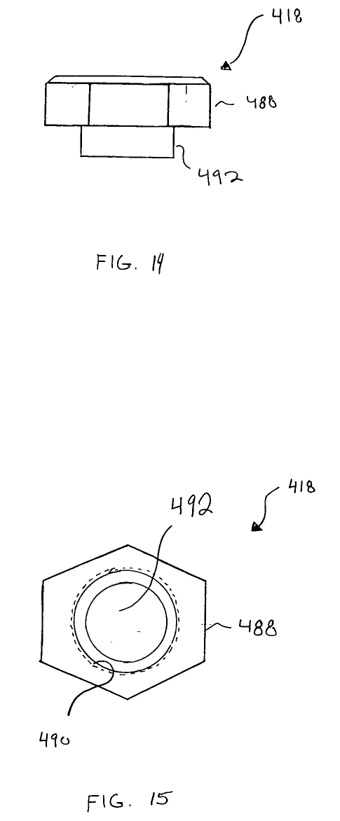

Referring to FIGS. 12 and 13, an alternative embodiment of a fastener is shown. Fastener 318 may include a set screw 380 and a cap 382. Set screw 380 may be externally threaded to engage internal threads formed on body 12, 112, 212 (described above). In addition, set screw 380 may include a recess 384 keyed to receive a driving tool, such as a hex wrench, torx wrench, or other tool known in the art. Cap 382 preferably includes an outer rim 386 that fits over the upper portion of body 12, 112, 212 (described above). Outer rim 386 may aid in preventing the upper portion of body 12, 112, 212 from splaying outward under the axial forces of set screw 380 when fastener 380 is tightened against a spinal rod received within the body 12, 112, 212. Set screw 380 and cap 382 may be formed integrally, or alternatively, may be separate pieces that may be joined by welding, bonding, press fitting or other techniques known in the art.

Referring to FIGS. 14 and 15, another alternative embodiment of a fastener is shown. According to this embodiment, fastener 418 is a nut 488 having internal threads 490 for engaging external threads formed on an upper surface of a body member (not shown). Fastener 418 may also include an internal spacer 492 to be received within the upper portion of a body member. Internal spacer 492, if provided, may help prevent the upper portion of a body member from deflecting inward under the axial forces applied by nut 488 when fastener 418 is tightened against a spinal rod. Nut 488 and spacer 492 may be formed integrally, or alternatively, may be separate pieces that may be joined by welding, bonding, press fitting or other techniques known in the art.

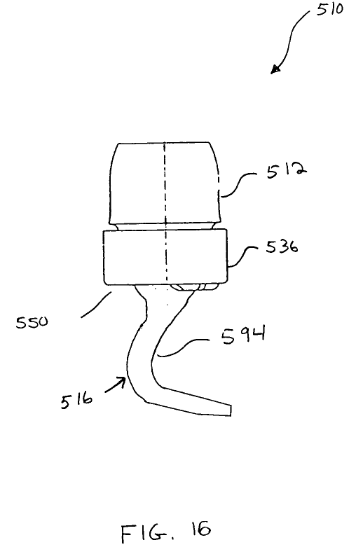

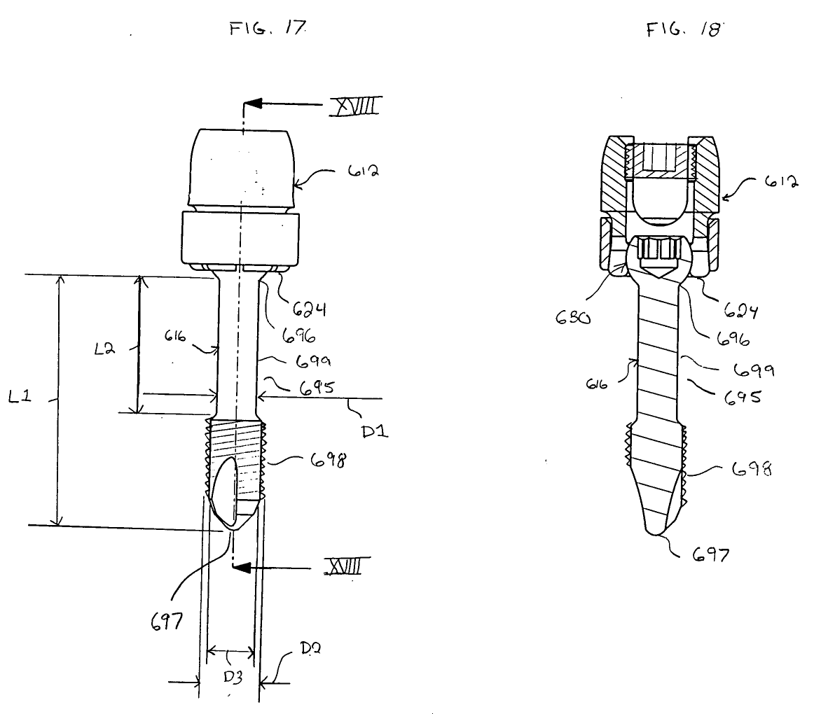

Referring to FIG. 16, an alternative embodiment of a polyaxial bone anchor 510 is shown in which anchor member 516 is a hook 594. According to this embodiment, hook 594 may be dimensioned and configured for attachment to a pedicle, lamina, or other portion of the vertebra, as known by one of ordinary skill in the art.

Referring to FIGS. 17 and 18, another alternative embodiment of a polyaxial bone anchor is shown. Polyaxial bone anchor 610 is substantially similar to polyaxial bone anchor 10 (described above and shown in FIGS. 1-7), except as detailed below. As shown in FIGS. 17 and 18, anchor member 616 may comprise a bone screw having a shank 695 with a first end 696 attached to curvate head 630 and a second end 697 opposite the first end 696. Additionally, shank 695 may include a threaded portion 698 and an unthreaded portion 699. As shown, unthreaded portion 699 may be substantially adjacent first end 696, and/or threaded portion 698 may be substantially adjacent second end 697, although other configurations are possible. Unthreaded portion 699 may help eliminate thread interference with nerve roots when anchor member 616 is implanted in a vertebra.

As shown in FIG. 17, shank 695 may define a shank length L1 from first end 696 to second end 697, and unthreaded portion 699 may define an unthreaded length L2. According to one preferred embodiment, unthreaded length L2 is greater than approximately ¼ of shank length L1. According to another preferred embodiment, unthreaded length L2 may be greater than approximately ½ of shank length L1.

Still referring to FIG. 17, unthreaded portion 699 may define an unthreaded outer diameter D1 and threaded portion 699 may define an outer thread diameter D2 that is greater than unthreaded outer diameter D1. Also, threaded portion 699 may define an inner thread diameter D3, with unthreaded outer diameter D1 being greater than inner thread diameter D3. Alternatively, D1 may be equal to or greater than D2.

It should be noted that in FIGS. 17 and 18, body 612 is not provided with a countersunk region 650 or other recessed area in its lower bounding edge 624. As a result, anchor member 616 may angulate equally with respect to body member 612 regardless of the orientation of anchor member 616 with respect to body member 612. For example, anchor member 616 may angulate through up to about 30° with respect to body 612 about all axes. One of ordinary skill in the art will appreciate, however, that a countersunk region may alternatively be provided in order to suit a specific medical application. One of ordinary skill in the art will also appreciate that body 612 may be used in the embodiments of FIGS. 1-16.

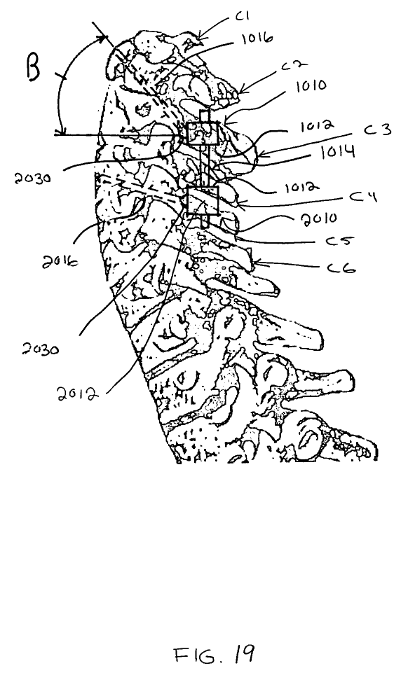

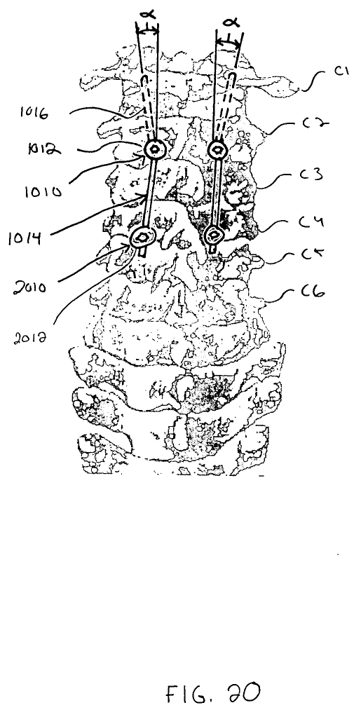

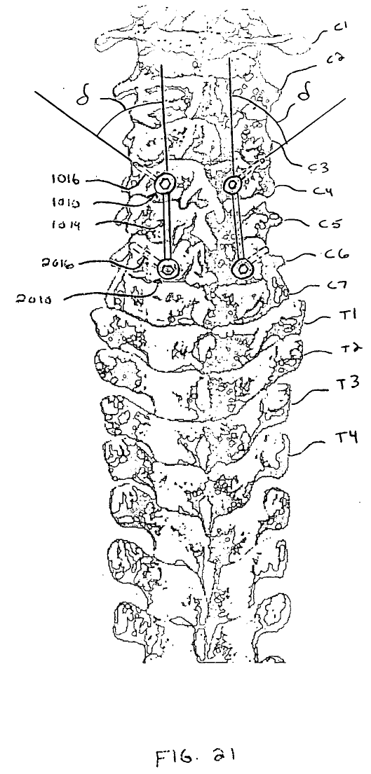

With reference to FIGS. 19 and 20, a first illustrative method of fixation of the cervical region of the spine will be described. The method described below may be performed using any of the polyaxial bone anchors described above, or any other polyaxial bone anchors known in the art, although the polyaxial bone anchors described above are preferred. The method generally includes the steps of attaching a first polyaxial bone anchor 1010 to the C1 and C2 vertebrae, preferably attaching a second polyaxial bone anchor 2010 to the C3 or C4 vertebra (although the C3 to T3 vertebrae are further possible alternatives), and securing a spinal rod to the first and second polyaxial bone anchors 1010, 2010 to align the vertebrae. This may be accomplished, for example, by inserting the bone screw 1016 of first polyaxial anchor 1010 through the caudal articular process of the C2 vertebra and into the lateral mass of the C1 vertebra, thereby immobilizing the C1 vertebra with respect to the C2 vertebra. The second bone anchor 2010 may alternatively be implanted into one or more vertebrae in other regions of the spine (i.e., the lower thoracic or lumbar regions).

In order to insert bone screw 1016 through the C2 vertebra and into the C1 vertebra, it may be necessary to insert bone screw 1016 at an orientation of between about 0° and about 25° medially or laterally, as represented by the angle α of FIG. 20, and more preferably between about 0° and about 15° medially or laterally. Additionally or alternatively, it may be necessary to insert bone screw 1016 at an orientation of between about 30° and about 50° upward, as represented by the angle β of FIG. 19, and more preferably between about 30° and about 40° upward. The countersunk regions described above with respect to the polyaxial bone anchors of the present invention may be configured and dimensioned to provide the necessary medial or lateral and/or upward angulation, although the present method is not limited to the structures of polyaxial bone anchors described herein.

Prior to inserting bone screw 1016, it may be desirable to drill and/or tap a hole from the C2 vertebra to the C1 vertebra. In the case where the hole is tapped, it may be preferable not to tap the anterior cortex of the C1 vertebra. Once bone screw 1016 has been fully inserted into the C2 and C1 vertebrae, the body 1012 may be snapped onto the curvate head 1030 of bone screw 1016. Alternatively, body 1012 and curvate head 1030 may be preassembled before bone screw 1016 is inserted into the C2 and C1 vertebrae.

Second polyaxial anchor 2010 is preferably attached to the C3 or C4 vertebra, for example, by threading bone screw 2016 into the C3 or C4 vertebra. Alternatively second polyaxial anchor 2010 may be attached to other vertebrae including those in the C3 to T3 range. Once the second polyaxial anchor 2010 is implanted, body 1012 and body 2012 may be rotated to align their respective rod-receiving channels (not illustrated in FIGS. 19 and 20) so that a spinal rod 1014 may be inserted therein. Once the vertebrae have been repositioned to correct the deformity at hand, the fasteners (not illustrated in FIGS. 19 and 20) of first and second polyaxial anchors 1010, 2010 may be tightened to secure the spinal rod 1014 to the first and second polyaxial anchors 1010, 2010, and to fix the angular positions of the bodies 1012, 2012 with respect to the bone screws 1016, 2016, thus forming a substantially rigid construct.

Alternatively, one end of the spinal rod 1014 can be inserted into one of the bodies 1012, 2012, and the spinal rod 1014 manipulated to reposition the vertebral bodies. Then the other end of the spinal rod 1014 can be inserted into the other of the bodies 1012, 2012 and then the spinal rod 1014 fixed in position. The first end of the spinal rod 1014 may be fixed in one of the bodies 1012, 2012 and the fastener fixed with respect to the body 1012, 2012 before the spinal rod 1014 is manipulated to reposition the vertebral bodies. In yet another embodiment of this method, the bone anchors 1010, 2010 may be inserted into the spine as described above, both ends of the spinal rod 1014 may be inserted into the anchors 1010, 2010 and one end of the spinal rod fixed or secured into the anchor 1010, 2010 and a distraction or compression force applied to move the polyaxial anchor along the spinal rod 1014 to apply either a distraction or compression force, and thereafter fixing the second end of the spinal rod 1014 into the polyaxial anchor.

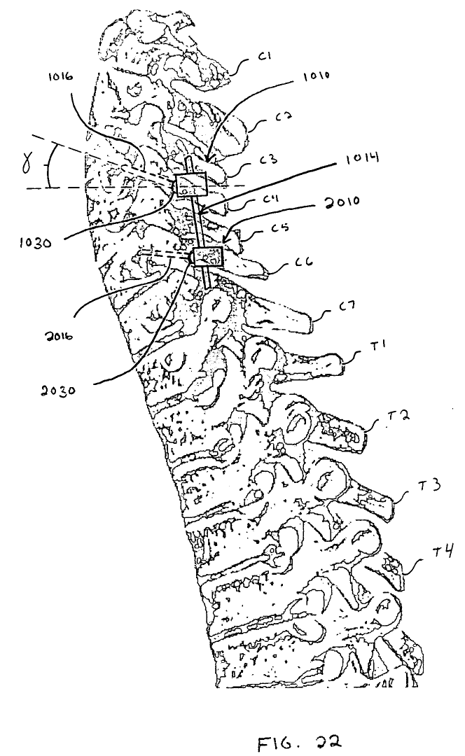

With reference to FIGS. 21 and 22, a second illustrative method of fixation of the cervical spine will be described. According to this method, a first bone screw 1016 may be inserted into the lateral mass of a first vertebra. For example, first bone screw 1016 may be inserted into any vertebra in the range from C3 to T3, for example, such as C4 as shown in FIGS. 21 and 22. Additionally, a second bone screw 2016 may be inserted into the lateral mass of a second vertebra. For example, second bone screw 2016 may be inserted through any other vertebra in the range from C3 to T3, for example, such as C6 as shown in FIGS. 21 and 22. Alternatively, the second bone screw 2016 may be implanted into one or more vertebrae in other regions of the spine (i.e., the lower thoracic or lumbar regions). As shown in FIGS. 21 and 22, the first and second bone screws 1016, 2016 may extend into the lateral mass of one vertebra only, or alternatively may extend into an adjacent vertebrae to fix the vertebrae together (e.g., as described above with respect to FIGS. 19 and 20).

It may be desirable to pre-drill and/or pre-tap holes in the vertebrae before implanting the bone screws. In the case where the holes are tapped, it may be preferable to tap only the proximal cortex. Also, bone screws 1016 and/or 2016 may be pre-assembled to bodies 1012, 2012 prior to implantation, or alternatively, the bodies 1012, 2012 may be snapped onto the curvate heads 1030, 2030 of the bone screws 1016, 2016 after the screws have been implanted.

In order to insert first bone screw 1016 or second bone screw 2016 into the lateral mass of the vertebra, it may be necessary to insert first or second bone screw 1016, 2016 at an orientation of between about 0° and about 50° upward, and preferably between about 25° and about 45° upward, as represented by the angle γ of FIG. 21. Additionally or alternatively, it may be necessary to insert first or second bone screw 1016, 2016 at an orientation of between about 0° and about 45° laterally, as represented by the angle δ of FIG. 22. According to one preferred embodiment, the starting point for the insertion of first bone screw 1016 or second bone screw 2016 is about 2 mm medial or about 2 mm medial and 2 mm caudal to the center of the lateral mass.

Once the first and second polyaxial anchors 1010, 2010 have been implanted, their bodies 1012, 2012 may rotated to align their respective rod-receiving channels (not illustrated in FIGS. 21 and 22) so that a spinal rod 1014 may be inserted therein. Once the vertebrae have been repositioned to correct the deformity at hand, the fasteners (not illustrated in FIGS. 21 and 22) may be tightened to secure the spinal rod 1014 to the first and second polyaxial anchors 1010, 2010, and to fix the angular positions of the bodies 1012, 2012 with respect to the bone screws 1016, 2016, thus forming a substantially rigid construct.

While it is apparent that the illustrative embodiments of the invention herein disclosed fulfill the objectives stated above, it will be appreciated that numerous modifications and other embodiments may be devised by those skilled in the art. Therefore, it will be understood that the appended claims are intended to cover all such modifications and embodiments which come within the spirit and scope of the present invention.

1. A polyaxial bone anchor for attaching a rod to a bone comprising:
an anchor member for attachment to the bone, the anchor member having a curvate head;
a body member polyaxially mounted on the curvate head, the body member defining a first axis;
a seat for orienting the rod along a second axis; and
a fastener capable of engaging the body member to press the rod against the seat;
wherein the first axis is oriented at an acute angle with respect to the second axis.

2. The polyaxial bone anchor of claim 1, wherein the first axis is oriented at an angle of between about 40° and about 60° with respect to the second axis.

3. The polyaxial bone anchor of claim 1, wherein the first axis is oriented at an angle of between about 45° and about 70° with respect to the second axis.

4. The polyaxial bone anchor of claim 1, further comprising an insert member disposed within the body member and having a compressible recess for receiving the head, wherein the seat is associated with the insert member.

5. The polyaxial bone anchor of claim 4, wherein the seat defines an inclined surface on the insert member, and the inclined surface extends substantially parallel to the second axis.

6. The polyaxial bone anchor of claim 4, wherein tightening the fastener presses the rod against the seat and causes the insert to compress around the curvate head and fix the angular position of the anchor member with respect to the body member.

7. The polyaxial bone anchor of claim 1, further comprising a collar disposed around the body member, wherein the seat is associated with the collar.

8. The polyaxial bone anchor of claim 7, wherein the seat defines an inclined surface on the collar, and the inclined surface extends substantially parallel to the second axis.

9. The polyaxial bone anchor of claim 7, wherein tightening the fastener presses the rod against the seat and causes the collar to compress the body around the curvate head and fix the angular position of the anchor member with respect to the body member.

10. The polyaxial bone anchor of claim 1, wherein the fastener is a set screw capable of engaging internal threads formed on the body member.

11. The polyaxial bone anchor of claim 10, further comprising an external cap associated with the set screw.

12. The polyaxial bone anchor of claim 1, wherein the fastener is a nut capable of engaging external threads formed on the body member.

13. The polyaxial bone anchor of claim 12, further comprising an internal spacer associated with the nut.

14. The polyaxial bone anchor of claim 1, wherein the anchor member is a screw or a hook.

15. The polyaxial bone anchor of claim 1, wherein the anchor member comprises a bone screw having a shank with a first end attached to the curvate head and a second end opposite the first end, and the shank includes an unthreaded portion and a threaded portion.

16. The polyaxial bone anchor of claim 15, wherein the unthreaded portion is substantially adjacent the first end, and the threaded portion is substantially adjacent the second end.

17. The polyaxial bone anchor of claim 16, wherein the shank defines a shank length from the first end to the second end, and the unthreaded portion extends over greater than about ¼ of the shank length.

18. The polyaxial bone anchor of claim 17, wherein the unthreaded portion extends over greater than about ½ of the shank length.

19. The polyaxial bone anchor of claim 15, wherein:
the unthreaded portion defines an unthreaded outer diameter;
the threaded portion defines an inner thread diameter and an outer thread diameter; and
the outer thread diameter is greater than the unthreaded outer diameter.

20. The polyaxial bone anchor of claim 19, wherein the unthreaded outer diameter is greater than the inner thread diameter.

21. The polyaxial bone anchor of claim 1, wherein the body member has a bore extending therethrough, and the bore defines the first axis.

22. The polyaxial bone anchor of claim 1, wherein the bone is a vertebra.

23. The polyaxial bone anchor of claim 1, wherein the rod is a spinal rod.

24. A polyaxial bone anchor for attaching a rod to a bone comprising:
an anchor member for attachment to the bone, the anchor member having a head;
a body member having a U-shaped channel for receiving the rod and a compressible recess for receiving the head such that the anchor member can initially polyaxially angulate with respect to the body member;
a collar slidably disposed about the body member and capable of compressing the recess around the head; and
a fastener capable of pressing the rod against the collar;
wherein the body member defines a first axis, an upper bounding edge, and a lower bounding edge, and the lower bounding edge includes a countersunk region to permit increased angulation of the anchor member with respect to the first axis when the anchor member is oriented toward the countersunk region.

25. The polyaxial bone anchor of claim 24, wherein the bounding edge is configured and dimensioned to permit the anchor member to angulate through a first angle of about 30° with respect to the first axis, and the countersunk region is configured and dimensioned to permit the anchor member to angulate through a second angle of about 50° with respect to the first axis.

26. The polyaxial bone anchor of claim 25, wherein the first angle is about 20° and the second angle is about 45°.

27. The polyaxial bone anchor of claim 24, wherein the U-shaped channel defines a second axis, and a midpoint of the countersunk region is offset from the second axis by between about 0° and about 45°.

28. The polyaxial bone anchor of claim 27, wherein the midpoint of the countersunk region is offset from the second axis by between about 20° and about 25°.

29. The polyaxial bone anchor of claim 24, wherein the countersunk region extends through an angular region of between about 5° and about 180° with respect to the first axis.

30. The polyaxial bone anchor of claim 24, wherein the countersunk region extends through an angular region of between about 15° and about 20° with respect to the first axis.

31. The polyaxial bone anchor of claim 24, wherein at least a portion of the body member has a tapered exterior surface, and at least a portion of the collar has a tapered interior surface.

32. The polyaxial bone anchor of claim 31, wherein sliding the collar downward with respect to the body member causes the tapered interior surface to engage the tapered exterior surface to compress the recess around the head to fix the orientation of the anchor member with respect to the body member.

33. The polyaxial bone anchor of claim 32, wherein tightening the fastener presses the rod against the collar to slide the collar downward with respect to the body member.

34. The polyaxial bone anchor of claim 24, wherein the fastener is a set screw capable of engaging internal threads formed on the body member.

35. The polyaxial bone anchor of claim 34, further comprising an external cap associated with the set screw.

36. The polyaxial bone anchor of claim 24, wherein the fastener is a nut capable of engaging external threads formed on the body member.

37. The polyaxial bone anchor of claim 36, further comprising an internal spacer associated with the nut.

38. The polyaxial bone anchor of claim 24, wherein the anchor member is a screw or a hook.

39. The polyaxial bone anchor of claim 24, wherein the anchor member comprises a bone screw having a shank with a first end attached to the head and a second end opposite the first end, and the shank includes an unthreaded portion and a threaded portion.

40. The polyaxial bone anchor of claim 39, wherein the unthreaded portion is substantially adjacent the first end, and the threaded portion is substantially adjacent the second end.

41. The polyaxial bone anchor of claim 40, wherein the shank defines a shank length from the first end to the second end, and the unthreaded portion extends over greater than about ¼ of the shank length.

42. The polyaxial bone anchor of claim 41, wherein the unthreaded portion extends over greater than about ½ of the shank length.

43. The polyaxial bone anchor of claim 39, wherein:
the unthreaded portion defines an unthreaded outer diameter;
the threaded portion defines an inner thread diameter and an outer thread diameter; and the outer thread diameter is greater than the unthreaded outer diameter.

44. The polyaxial bone anchor of claim 43, wherein the unthreaded outer diameter is greater than the inner thread diameter.

45. The polyaxial bone anchor of claim 24, wherein the bone is a vertebra.

46. The polyaxial bone anchor of claim 24, wherein the rod is a spinal rod.

47. The polyaxial bone anchor of claim 24, wherein the head is substantially spherical.

48. A method of fixating the cervical region of the spine using a first polyaxial bone anchor having a first screw member and a first body member with a first rod-receiving channel, and a second polyaxial bone anchor having a second screw member and a second body member with a second rod-receiving channel, the method comprising the steps of:
inserting the first screw member through a first vertebra and into a second vertebra;
inserting the second screw member into a third vertebra;
aligning the first rod-receiving channel with the second rod-receiving channel; and
securing a spinal rod in the first rod-receiving channel and in the second rod-receiving channel.

49. The method of claim 48, wherein the first screw member extends through a C2 vertebra and into a C1 vertebra.

50. The method of claim 49, wherein the first screw member extends into a lateral mass of the C1 vertebra.

51. The method of claim 49, wherein the first screw member extends through a caudal articular process of the C2 vertebra.

52. The method of claim 48, wherein the first screw member immobilizes the second vertebra with respect to the first vertebra.

53. The method of claim 48, wherein the first screw member is inserted at an orientation of between about 0° and about 25° medially or laterally.

54. The method of claim 48, wherein the first screw member is inserted at an orientation of between about 0° and about 15° medially or laterally.

55. The method of claim 48, wherein the first screw member is inserted at an orientation of between about 30° and about 50° upward.

56. The method of claim 48, wherein the first screw member is inserted at an orientation of between about 30° and about 40° upward.

57. The method of claim 48, wherein the step of inserting the first screw member comprises drilling a first hole from the first vertebra to the second vertebra.

58. The method of claim 57, wherein the step of inserting the first screw member further comprises tapping at least a portion of the first hole.

59. The method of claim 48, wherein the first body member defines a first axis, an upper bounding edge, and a lower bounding edge, and the lower bounding edge includes a countersunk region to permit increased angulation of the first screw member with respect to the first axis when the first screw member is oriented toward the countersunk region.

60. The method of claim 59, wherein the bounding edge is configured and dimensioned to permit the first screw member to angulate through a first angle of about 30° with respect to the first axis, and the countersunk region is configured and dimensioned to permit the first screw member to angulate through a second angle of about 50° with respect to the first axis.

61. The method of claim 60, wherein the first angle is about 20° and the second angle is about 45.

62. The method of claim 59, wherein the first rod-receiving channel defines a second axis, and a midpoint of the countersunk region is offset from the second axis by between about 0° and about 45°.

63. The method of claim 60, wherein the countersunk region extends through an angular region of between about 5° and about 180° with respect to the first axis.

64. A method of fixating the spine using a first polyaxial bone anchor having a first screw member and a first body member with a first rod-receiving channel, and a second polyaxial bone anchor having a second screw member and a second body member with a second rod-receiving channel, the method comprising the steps of:
inserting the first screw member into a lateral mass of a first vertebra;
inserting the second screw member into a second vertebra;
aligning the first rod-receiving channel with the second rod-receiving channel; and
securing a spinal rod in the first rod-receiving channel and in the second rod-receiving channel.

65. The method of claim 64, wherein the second screw member is inserted into a lateral mass of the second vertebra.

66. The method of claim 64, wherein at least one of the first and second vertebrae is selected from the group of vertebrae consisting of C3, C4, C5, C6, C7, T1, T2 and T3.

67. The method of claim 64, wherein the first screw member is inserted at an orientation of between about 0° and about 45° laterally.

68. The method of claim 64, wherein the first screw member is inserted at an orientation of between about 0° and about 50° upward.

69. The method of claim 64, wherein the first screw member is inserted at an orientation of between about 25° and about 45° upward.

70. The method of claim 64, wherein the first body member defines a first axis, an upper bounding edge, and a lower bounding edge, and the lower bounding edge includes a countersunk region to permit increased angulation of the first screw member with respect to the first axis when the first screw member is oriented toward the countersunk region.

71. The method of claim 70, wherein the bounding edge is configured and dimensioned to permit the first screw member to angulate through a first angle of about 30° with respect to the first axis, and the countersunk region is configured and dimensioned to permit the first screw member to angulate through a second angle of about 50° with respect to the first axis.

72. The method of claim 71, wherein the first angle is about 20° and the second angle is about 45°.

73. The method of claim 70, wherein the first rod-receiving channel defines a second axis, and a midpoint of the countersunk region is offset from the second axis by between about 0° and about 45°.

74. The method of claim 70, wherein the countersunk region extends through an angular region of about 5° and about 180° with respect to the first axis.
